(19) United States
(12) Patent Application Publication
(10) Pub. No.: US 2026/0069279 A1
(43) Pub. Date: Mar. 12, 2026

(54) TUBULAR BRAIDED IMPLANTABLE ENDOVASCULAR EMBOLIZATION DEVICE

(71) Applicant: DePuy Synthes Products, Inc., Raynham, MA (US)

(72) Inventors: Lacey GOROCHOW, Miami, FL (US); Daniel SOLAUN, Miami, FL (US); Pedro D. PEDROSO, Parkland, FL (US); Patrick BROUWER, Haarlem (NL)

(73) Assignee: DePuy Synthes Products, Inc., Raynham, MA (US)

(21) Appl. No.: 18/828,484

(22) Filed: Sep. 9, 2024

(51) Int. Cl. A61B 17/12; A61B 90/00

(52) U.S. Cl. CPC A61B 17/12172; A61B 17/12113; A61B 2017/1205; A61B 2090/3983

(57) ABSTRACT

Tubular braided implantable endovascular embolization device including a folded over tubular braid forming a folded over atraumatic distal surface and preformed into multiple distinct sections including: a stabilizing section; a sealing section; an intermediate section interposed between the sealing and stabilizing sections; and a proximal section. The stabilizing section and the sealing section are radially self-expanding to a maximum outer diameter larger than the intermediate section or the proximal section. Enhanced stiffness while obstructing open space regions maximizing disruption of blood flow therethrough is achieved by using a plurality of nested layers (at least one of which is the folded over tubular braid) to form the sealing section while the stabilizing section preferably formed by a single braided layer (i.e., the folded over tubular braid) optimizes compressibility and positioning.

### FIELD

The present disclosure relates to an endovascular medical treatment device, and more particularly, to a tubular braided implantable endovascular embolization device to disrupt blood flow at the target site. By way of non-limiting example, the tubular braided implantable endovascular embolization device may be used in the treatment of an aneurysm to divert (i.e., impede) blood flow or to restrict blood flow in a vessel (e.g., in the brain or peripheral vasculature).

### BACKGROUND

An aneurysm stretches out thereby thinning a section of the wall of the artery. Cranial aneurysms may be difficult to treat due to their proximity to critical brain tissues. Conventional solutions have included endovascular treatment whereby an internal volume of the aneurysm sac is surgically removed or excluded from arterial blood pressure and flow via an endovascular intrasaccular device. Current alternatives to endovascular intrasaccular devices or other surgical approaches include endovascularly delivered treatment devices that fill the sac (i.e., dome) of the aneurysm with embolic material or block the entrance (i.e., neck) of the aneurysm. Both approaches attempt to prevent or divert blood flow into the aneurysm. By filling an aneurysm sac, the embolic material clots the blood, creating a thrombotic mass within the aneurysm. Whereas, treating the aneurysm neck, blood flow into the entrance of the aneurysm is impeded, inducing venous stasis in the aneurysm and facilitating a natural formation of a thrombotic mass within the aneurysm.

Current intravascularly delivered implantable embolization devices typically utilize multiple devices (e.g., embolic coils) to either fill the sac or treat the entrance (i.e., neck) of the aneurysm. Naturally formed thrombotic masses created by treating the entrance with embolic coils may result in improved healing compared to aneurysm masses packed with embolic coils because naturally formed thrombotic masses can reduce the likelihood of distention from arterial walls and facilitate reintegration into the original parent vessel shape along the neck plane. However, embolic coils delivered to the neck of the aneurysm can potentially have the adverse effect of impeding the flow of blood in the adjoining blood vessel, particularly if the entrance is overpacked. Conversely, if the entrance is insufficiently packed, recanalization of blood flow may persist into the aneurysm. Treating certain aneurysm morphology (e.g., wide neck, bifurcation, etc.) may require ancillary devices (e.g., stents or balloons) to support the coil mass and obtain the desired packing density. Once implanted, the coils cannot easily be retracted or repositioned. Furthermore, aneurysms treated with multiple coils over time often recanalize or compact resulting from poor coiling, lack of coverage across the aneurysm neck, blood flow, and/or relatively large aneurysm size.

Alternatives to embolic coils are being explored, for example a tubular braided implant as disclosed in U.S. Pat. Nos. 10,653,425; 10,751,066; 11,278,292; 11,413,046; and 11,583,282, each of which is incorporated herein by reference in their entirety. Tubular braided implants have the potential to easily, accurately, and safely treat an aneurysm or other arterio-venous malformation in a parent vessel without blocking flow into perforator vessels communicating with the parent vessel. Compared to embolic coils, however, tubular braided implants are a newer technology, and there is therefore capacity for improved geometries, configurations, delivery systems, optimization of disruption of blood flow, etc. Regarding the geometry, it is desirable to design the tubular braided implant to minimize risk of damage to the vessel wall. This is a factor in all vasculature treatment procedures, but particularly significant during treatment of an aneurysm in which the vessel wall is inherently thin. Several factors contribute to optimizing disruption of blood flow. During delivery through the microcatheter, conventional tubular braided implants may undesirably twist in configuration relative to the axial/longitudinal axis extending therethrough. In a twisted orientation the efficiency of the implanted conventional tubular braided device to disrupt blood flow to the aneurysm is compromised. Despite most likely being unsuccessful, attempts may be made to untwist the conventional tubular braided implant by torquing the delivery wire after the implanted conventional tubular braided device has exited from the catheter (i.e., in a deployed/implanted state at the target site in the vessel of the patient). If not successfully untwisted while implanted, then the twisted conventional tubular braided implant must be fully withdrawn from the microcatheter. Once outside the body, the twisted conventional tubular braided implant may be manipulated by hand to its original untwisted orientation prior to reattempting delivery of the same device. Otherwise, following withdraw of the twisted conventional tubular braided implant from the body, a new tubular braided implantable device may be delivered via the microcatheter to the target site. Efficiency of diversion of blood flow may also be optimized by preventing or minimizing probability of migration over time of the tubular braided implanted device (i.e., future recanalization).

It is therefore desirable to develop an improved single (e.g., assembled as a single unit or piece, i.e., a unitary structure) tubular braided implantable endovascular embolization device that addresses all these factors.

### SUMMARY

An aspect of the present disclosure relates to an improved tubular braided implantable endovascular embolization device having a folded over atraumatic distal surface that prevents or minimizes risk of damage to the vessel wall during deployment (i.e., implantation after exiting from the microcatheter).

Another aspect of the present disclosure is directed to an improved tubular braided implantable endovascular embolization device having a folded over atraumatic distal surface serving as a bumper while repositioning the sealing section to be properly seated at the neck of the aneurysm.

Yet another aspect of the present disclosure is directed to an improved tubular braided implantable endovascular embolization device comprising a stabilizing section apposing the aneurysm wall so as to be anchored in place while working in tandem with the intermediate section imposing a force against and stabilizing at the aneurysm neck the sealing section thereby minimizing migration distally over time (i.e., reducing risk of future recanalization).

While another aspect of the present disclosure relates to an improved tubular braided implantable endovascular embolization device with multiple distinct sections of varying stiffness (i.e., rigidity) to minimize risk of damage to the aneurysm wall, maximize compressibility and positioning, and optimize disruption of blood flow. Preferably, of the multiple distinct sections the stabilizing section has a lowest stiffness (i.e., rigidity), the intermediate section having increased stiffness or rigidity relative to the stabilizing section, and the sealing section exhibiting a highest stiffness (i.e., rigidity) compared to that of the intermediate section.

Still another aspect of the present disclosure relates to an improved tubular braided implantable endovascular embolization device including respective stabilizing and sealing sections that when deployed (i.e., upon exiting from the distal end/tip of the microcatheter) each section self-expands radially to an implanted size and shape dependent on the anatomy (e.g., size and shape) of the aneurysm in which it is implanted, while and intermediate section disposed therebetween has a smaller outer diameter relative to either of the stabilizing or sealing sections while in the radially self-expanded state.

While another aspect of the present disclosure is directed to an improved tubular braided implantable endovascular embolization device that at all times, and in particular, during delivery through the delivery device (e.g., microcatheter)) and upon exiting from the distal end thereof, is preferably independently freely rotatable relative to the pushing device (e.g., delivery wire) minimizing or preventing twisting in a longitudinal/axial direction thereby optimizing efficiency of diversion of blood flow when deployed at the target site in its natural, default, or original (i.e., non-twisted) configuration.

Another aspect of the present disclosure relates to an improved tubular braided implantable endovascular embolization device independently freely rotatable relative to the delivery wire both during delivery through the microcatheter and upon exiting from the distal end thereof, thereby minimizing twisting and resulting built up energy therefore decreasing the track force allowing delivery via smaller size microcatheters.

Still another aspect of the present disclosure relates to an improved tubular braided implantable endovascular embolization device that when the stabilizing section is deployed at the target site (e.g., in the sac/dome of the aneurysm) substantially centers the microcatheter off the aneurysm wall.

In yet still another aspect the present disclosure relates to an improved tubular braided implantable endovascular embolization device wherein the intermediate section acts as a hinge permitting off angle positioning of the sealing section at the neck of the aneurysm relative to that of the stabilizing section while anchored in place in the sac/dome of the aneurysm, such aspect being particularly well suited for treatment of off angle aneurysms.

Another aspect of the present disclosure relates to an improved tubular braided implantable endovascular embolization device including a multi-layer sealing section optimizing disruption (e.g., diversion or occlusion) of blood flow passable though open space regions defined in the multi-layer sealing section, while maintaining maximum compressibility and positioning of the stabilizing section formed from only a single braided layer.

While still yet another aspect of the present disclosure is directed to an improved tubular braided implantable endovascular embolization device with substantially all (e.g., ≥approximately 95%) of the sealing section disposed in the aneurysm (e.g., in the sac/dome including the neck) with minimal (e.g., ≤approximately 5%) protrusion of the sealing section into the parent vessel.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices of the present disclosure, by way of example only, not by way of limitation.

FIG. 1A is a side view of an example of the tubular braided implantable endovascular embolization device in accordance with the present disclosure having multiple distinct sections including: a stabilizing section, an intermediate section, a sealing section, and a proximal section; wherein the sealing section includes two nested braided layers; the illustration further depicting a portion of the distal end of the delivery wire and longitudinal/axial cross-sectional view through the microcatheter;

FIG. 1B is a longitudinal/axial cross-sectional view along line 1B-1B through the tubular braided implantable endovascular embolization device of FIG. 1A; wherein the curved arrow denotes the free rotation of the embolization device relative to the delivery wire;

FIG. 1C is a schematic representation along the longitudinal/axial axis of the tubular braided implantable endovascular embolization device wherein the two nested braided layers of the sealing section includes a single outermost layer of the folded over tubular braid and separate therefrom a second supplemental structural obstructing layer (i.e., filler layer) (e.g., the innermost braided layer); wherein the free edges of the respective braided layers forming the embolization device are secured via an intermediate marker band disposed about the intermediate section and a proximal maker band disposed about the proximal section;

FIG. 1D is a schematic representation along the longitudinal/axial axis of another example of the tubular braided implantable endovascular embolization device wherein the sealing section includes two nested braided layers formed by the folded over tubular braided layer (e.g., outermost braided layer and the innermost braided layer); wherein the intermediate marker band is eliminated and the free edges of the respective braided layers forming the embolization device are secured via only a proximal maker band disposed about the proximal section;

FIG. 1E is an enlarged view of section 1E in FIG. 1A illustrating an exemplary independently freely rotatable connection of the tubular braided implantable endovascular embolization device relative to the delivery wire;

FIG. 1F is a radial cross-section view through the double layer sealing section in FIG. 1A while in a radially constricted state of minimum outer diameter (deliverable through the microcatheter to the target site) depicting the individual woven braided wires comprising each the two nested braided layers;

FIG. 2A is a side view of a second example of the tubular braided implantable endovascular embolization device in accordance with the present disclosure with multiple distinct sections including: a stabilizing section, an intermediate section, a sealing section, and a proximal section; wherein the sealing section includes three nested braided layers; the illustration includes a portion of the distal end of the delivery wire and longitudinal/axial cross-sectional view through the microcatheter;

FIG. 2B is a longitudinal/axial cross-sectional view along line 2B-2B through the tubular braided implantable endovascular embolization device of FIG. 2A; wherein the curved arrow denotes the free rotation of the embolization device relative to the delivery wire;

FIG. 2C is a schematic representation along the longitudinal/axial axis of the tubular braided implantable endovascular embolization device of FIG. 2A wherein the three nested braided layers of the sealing section includes the folded over tubular braided layer representing the two outer braided layers and a separate supplemental structural obstructing layer (e.g., third braided innermost layer); wherein the free edges of the respective braided layers forming the embolization device are secured via an intermediate marker band disposed about the intermediate section and a proximal maker band disposed about the proximal section;

FIG. 2D is a radial cross-section view through the triple braided layer sealing section in FIG. 2A while in a radially constricted state of minimum outer diameter (deliverable through the microcatheter to the target site) depicting the individual woven braided wires comprising each the three nested braided layers;

FIG. 2E is a schematic representation along the longitudinal/axial axis of another example of the tubular braided implantable endovascular embolization device wherein the three nested braided layers of the sealing section includes the folded over tubular braided layer representing the single outermost braided layer and separate therefrom two supplemental structural obstructing layers (e.g., second supplemental structural obstructing braided layer and third supplemental structural obstructing braided layer); wherein the free edges of the respective layers forming the embolization device are secured via an intermediate marker band disposed about the intermediate section and a proximal maker band disposed about the proximal section;

FIG. 2F is a schematic representation along the longitudinal/axial axis of yet another example of the tubular braided implantable endovascular embolization device wherein the three nested braided layers of the sealing section includes the folded over tubular braided layer representing the single outermost braided layer and separate therefrom two supplemental structural obstructing layers (e.g., supplemental structural obstructing folded over tubular braid); wherein the free edges of the respective layers forming the embolization device are secured via an intermediate marker band disposed about the intermediate section and a proximal maker band disposed about the proximal section;

FIG. 3 is an exemplary flow chart of use of the tubular braided implantable endovascular embolization device in accordance with the present disclosure;

FIGS. 4A-I depict sequential stages of deployment of the tubular braided implantable endovascular embolization device of FIG. 1A in the treatment of an aneurysm;

FIG. 5 is an exemplary flow chart of manufacture of the assembled tubular braided implantable endovascular embolization device in accordance with the present disclosure; and

FIG. 6A-6I depict sequential stages of deployment of the tubular braided implantable endovascular embolization device of FIG. 1A in impeding or restricting blood flow to a vessel in the brain.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

The present disclosure is directed to a single device for diversion/disruption of blood flow, e.g., diversion of blood flow to an aneurysm or impeding blood flow in a vessel such as in the brain or peripheral vasculature. During treatment of an aneurysm, rather than employing the time-consuming process of packing the sac/dome of the aneurysm with multiple embolization devices (e.g., multiple conventional embolic coils typically of varying size), the delivery and deployment procedure in accordance with the present disclosure is streamlined to only a single assembled tubular braided implantable endovascular embolization device. In addition, the tubular braided implantable endovascular embolization device is self-adjusting to an implanted state having a size and shape dependent on (i.e., conforming to) the anatomy of aneurysms of varying size and shape. Also, the tubular braided implantable endovascular embolization device in accordance with the present disclosure advantageously: (i) minimizes risk of damage to the vessel wall; (ii) maximizes compressibility and positioning at the target site; (iii) maximizes diversion (i.e., impeding) of blood flow away from an aneurysm or restricting (i.e., impeding) blood flow in a vessel (e.g., in the brain or peripheral vasculature); and (iv) minimizes risk of migration in a distal direction over time hence reducing probability of future recanalization.

The tubular braided implantable endovascular embolization device is formed starting with a tubular braid made of a plurality of wires woven into a desired pattern (e.g., full diamond pattern). To maximize compressibility and minimize risk of damage to the vessel wall each of the plurality of wires has an outer diameter preferably ≤approximately 0.001″. The wires are made of a biocompatible memory shape material (e.g., Nitinol—Nickle titanium alloy) pre-formable (e.g., heat set) to have a natural, default, or original shape (i.e., radially expanded state of maximum outer diameter) when free from an externally applied radially compressive or radially constraining force, but radially compressible (i.e., collapsed state having a reduced outer diameter) when subject to the externally applied radially compressive or radially constraining force. Initially (prior to folding over itself), the tubular braid preferably forms a right cylinder having an inner passageway extending in an axial/longitudinal direction between a first free edge at a first open end and a second free edge at an opposite second open end. One free edge of the tubular braid is folded over itself (i.e., inverted on itself or socked over itself) to form a folded over atraumatic distal surface 105 and inner channel 140. No feature or part of the tubular braided implantable endovascular embolization device extends distally of the folded over atraumatic distal surface 105 representing its distal most feature. During deployment at the target site, the folded over atraumatic distal surface 105 prevents or minimizes risk of damage to the vessel wall, which in the treatment of an aneurysm is already thinner than normal and hence at heightened risk.

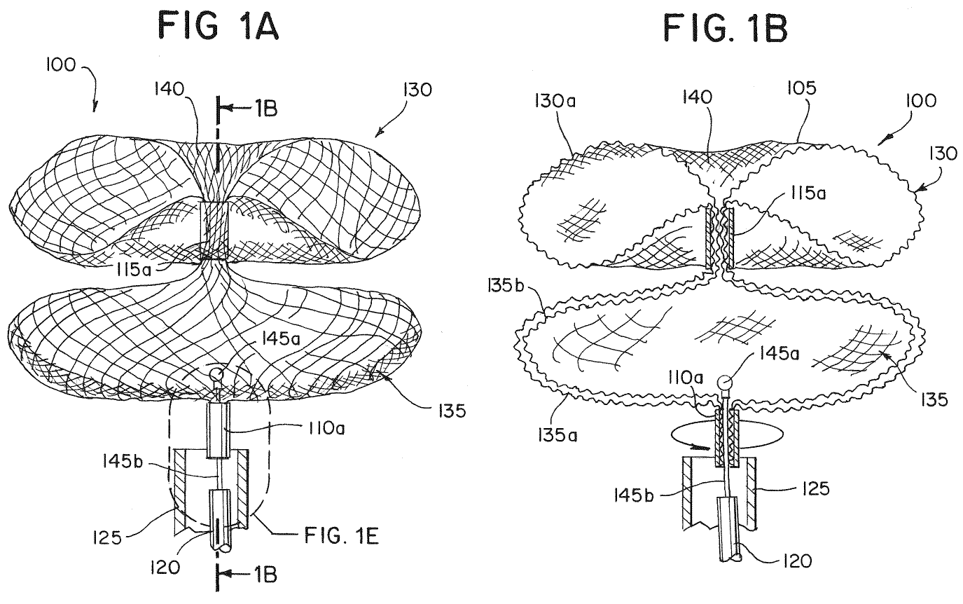

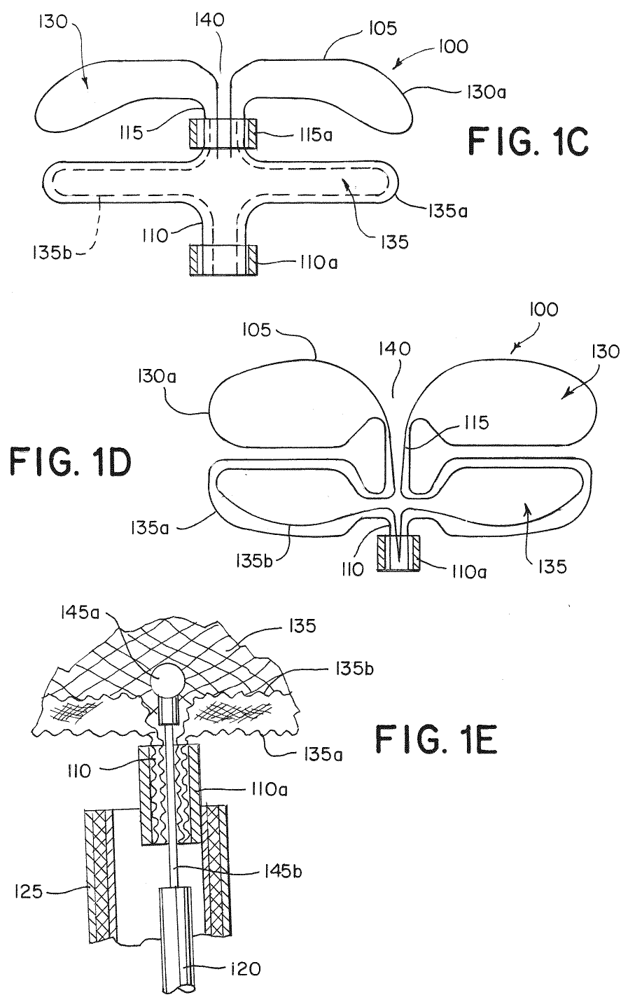

When folded over itself (i.e., inverted on itself or socked over itself) the respective first and second free edges of the tubular braid extend in the same proximal direction opposite that of (i.e., away from) the folded over atraumatic distal surface 105. The respective first and second free edges of the folded over tubular braid may either be substantially aligned or offset from one another in the longitudinal/axial direction. As shown in the example of FIG. 1D, the respective free edges substantially aligned with each other together represent the proximal end of the tubular braided implantable endovascular embolization device 100. In an alternative example in FIG. 1C, the respective free edges of the folded over tubular braid are offset from one another in the longitudinal/axial direction. With such offset configuration, one free edge of the folded over tubular braid forms the proximal end of the tubular braided implantable endovascular embolization device, while the other free edge of the folded over tubular braid is distally offset in the longitudinal/axial direction relative thereto.

With the two free edges arranged either substantially aligned or offset in the longitudinal/axial direction, the folded over tubular braid is pre-formed (i.e., heat set) into a natural, default, or original shape of multiple distinct sections each serving different functions during delivery and/or deployment. Starting from the atraumatic folded over distal surface 105 and extending to the opposite proximal end the tubular braided implantable endovascular embolization device, in series (one directly after the other), includes: a stabilizing section 130, an intermediate section 115, a sealing section 135, and a proximal section 110.

The stabilizing section 130 includes the folded over atraumatic distal surface 105 and represents a distal most section of the tubular braided implantable endovascular embolization device 100. As previously mentioned, the folded over distal surface 105 of the stabilizing section 130 provides an atraumatic (e.g., curved or rounded) surface preventing or minimizing risk of damage to the vessel wall (e.g., the sac/dome of the aneurysm) when deployed at the target site (e.g., aneurysm). No part or feature of the tubular braided implantable endovascular embolization device extends distally beyond the folded over atraumatic distal surface 105. As an additional benefit, when fully deployed in the sac/dome the folded over atraumatic distal surface 105 maximizes coverage of the wall for ruptured aneurysms. The stabilizing section 130 may be pre-formed (e.g., heat set) such that while in a natural, default, or original state has a predetermined radially self-expanding shape of maximum outer diameter. In a natural, default, or original state of maximum outer diameter the stabilizing section 130 may, for example, resemble that of a cap on a mushroom or acorn, be conically shaped, or torus shape. Other radially self-expanded shapes of the pre-formed stabilizing section 130 are contemplated that incorporate the folded over atraumatic distal surface 105.

To fulfill the goals of preventing or minimizing risk of damage to the vessel wall (i.e., minimizing stiffness or rigidity) while optimizing compressibility and positioning when deployed at the target site, the stabilizing section 130 is preferably formed by only a single braided layer 130a (i.e., single layer of the folded over tubular braid). In furtherance of these stated goals, individual wires forming the folded over tubular braid preferably each have an outer diameter≤approximately 0.001″. During delivery to the target site, the stabilizing section 130 is radially collapsed (i.e., reduced in outer diameter) while radially constrained within the lumen of the delivery device (e.g., microcatheter). Upon exiting from the distal end/tip of the microcatheter, the deployed stabilizing section 130 self-expands radially to an implanted size and shape dependent on the anatomy (e.g., size and shape) of the sac/dome of the aneurysm. When deployed, stabilizing section 130 apposes (i.e., directly imposing a force and pushing against) the aneurysm wall with sufficient force to remain anchored and stabilized in place. Due to its compressibility, radially and/or longitudinally, the stabilizing section 130 is self-adjusting to the implanted size and shape dependent on the anatomy (e.g., size and/or shape) of the particular aneurysm or vessel in which it is implanted. When anchored in place apposing the aneurysm wall the deployed stabilizing section 130 substantially centers the microcatheter relative thereto assisting during deployment of those remaining sections (e.g., intermediate section 115, sealing section 135, and proximal section 110) of the embolization device yet to exit the distal end/tip of the microcatheter.

Continuing in a proximal direction, the next section is the intermediate section 115 (i.e., bridge section or stalk section) having a narrow outer diameter that is: (i) smaller relative to that of the natural, default, original state radially expanded of maximum outer diameter of either the stabilizing section 130 or the sealing section 135 between which it is directly interposed; and (ii) less than or equal to the inner diameter of the lumen of the microcatheter through which the embolization device is deliverable to the target site. In one example configuration, regardless of the state of any other section of the embolization device at any given time, the narrow diameter of the intermediate section 115 is maintained at all times substantially unchanging in outer diameter (i.e., radially non-transitioning or not radially self-expanding). Such substantially unchanging aforementioned narrow outer diameter of the intermediate section 115 may be realized in several different ways. During manufacture the intermediate section may be pre-formed (e.g., heat set) as a radially non-transitioning (i.e., not radially self-expanding) region having the aforementioned narrow outer diameter. Alternatively, the intermediate section 115 having the aforementioned narrow outer diameter may be repositionable via a mechanical radially constraining device (e.g., intermediate marker band) freely slidable in a longitudinal/axial direction about a pre-formed radially self-expanding region of the folded over tubular braid interposed between the stabilizing section 130 and sealing section 135. During deployment of the embolization device (i.e., while exiting from the distal tip/end of the microcatheter), the freely slidable intermediate marker band automatically repositions itself in the longitudinal/axial direction, simultaneously self-adjusting in size the respective stabilizing section 130 and sealing section 135 dependent on the anatomy of the aneurysm. Due to its aforementioned narrow outer diameter, the intermediate section 115 is significantly more difficult to compress (i.e., substantially incompressible) longitudinally/axially and radially providing increased stiffness or rigidity relative to that of the stabilizing section 130 when deployed at the target site. The enhanced stiffness/rigidity of the intermediate section 115 interposed directly between the stabilizing section 130 and the sealing section 135 at its respective distal and proximal ends advantageously provides column strength. While the stabilizing section 130 remains anchored in place, the enhanced stiffness or rigidity of the intermediate section 115 imposes in a proximal direction a force on thereby stabilizing the sealing section 135 at the neck of the aneurysm minimizing risk of migration in a distal direction over time (i.e., future recanalization). Furthermore, intermediate section 115 acts like a hinge allowing independent offset angular positioning of the sealing section 135 relative to that of the stabilizing section 130 while anchored in place. Such offset angular positioning of the respective sealing section 135 relative to the stabilizing section 130 is particularly well suited for treatment of off angle aneurysms.

Continuing in a proximal direction, the next section of the embolization device is the sealing section 135. During manufacture of the embolization device, the sealing section 135 is pre-formed (i.e., heat set) in a natural, default, original state (i.e., radially expanded state) having a predetermined maximum outer diameter greater than or equal to the entrance (i.e., neck) of the aneurysm or inner diameter of the blood vessel to be restricted (e.g., occluded). During delivery the target site, the sealing section 135 while radially collapsed (i.e., reduced in outer diameter) is radially constrained within the lumen of the delivery device (e.g., microcatheter). Upon exiting from the distal end/tip of the microcatheter, the deployed sealing section 135 self-expands radially to an implanted size and shape dependent on the anatomy (e.g., size and shape) of the aneurysm (including the neck and the sac/dome). The deployed sealing section 135 is stabilized at the neck of the aneurysm via the force imposed by the intermediate section 115 working together with the stabilizing section 130 anchored in place in the sac/dome.

In the treatment of an aneurysm using an implantable embolization device it is desirable to maximize disruption or diversion of blood flow to optimize healing at the site of the neck of the aneurysm. Maximum disruption or diversion of blood flow may be realized by increasing the number of layers (e.g., more than one braided layer) forming the embolization device hence obstructing or impeding passage of blood thorough the openings defined by the woven braided wires. However, additional layers undesirably hamper compression and positioning of the embolization device in the aneurysm. Both conflicting factors are addressed in the tubular braided implantable endovascular embolization device in accordance with the present disclosure. To promote healing by maximizing disruption or diversion of blood flow the sealing section 135 positionable at the neck of the aneurysm includes multiple layers (e.g., two braided layers or three braided layers) nested together one inside the other, while the stabilizing section 130 having only a single layer (e.g., single braided layer) remains unhampered in both compressibility and positioning. This increase in material provided by the nested multiple layers (e.g., two braided layers or three braided layers) forming the sealing section obstruct or impede (e.g., reducing in size) open space regions defined by the nested multiple layers thereby maximizing disruption without completely/totally prohibiting passage of blood flow therethrough (i.e., more efficient diversion of blood flow away from the aneurysm). Inventive features associated with distinct sections of the tubular braided implantable endovascular embolization device operating together stabilize the sealing section 135 at the neck of the aneurysm minimizing risk of migration in a distal direction over time (i.e., future recanalization). In particular, stabilization at the neck of the aneurysm is maximized by the enhanced stiffness of the multi-layer sealing section 135 in combination with a force imposed thereon in a proximal direction towards the neck of the aneurysm (i.e., away from the folded over atraumatic distal surface 105) by the intermediate section 115 (fostered by its heightened stiffness) while being supported by the stabilizing section 130 anchored in place.

Upon exiting from the distal end/tip of the microcatheter into the sac/dome of the aneurysm, the sealing section 135 automatically self-expands radially to its implanted size and shape dependent on the anatomy (e.g., size and shape) of the aneurysm. While visible under imagery (e.g., fluoroscopic imagery), by physically manipulating the delivery wire 120 (e.g., partially withdrawing in a proximal direction and/or advancing in a distal direction) the sealing section 135 is repositionable to be properly seated at the neck of the aneurysm. Risk of damage to the aneurysm wall during repositioning of the embolization device is minimized by the folded over atraumatic distal surface 105 acting as a bumper. When properly seated at the neck of the aneurysm, substantially all (i.e., approximately ≥95%) of the sealing section 135 is disposed within the aneurysm (including both the sac/dome and neck) while only a minimal portion (e.g., ≤5%) protrudes or extends beyond the neck of the aneurysm into the adjacent parent vessel.

The most proximal part of the tubular braided implantable endovascular embolization device is the proximal section 110. During manufacture the proximal section 110 has a pre-formed radially non-transitioning narrow outer diameter that is: (i) smaller relative to that of the natural, default, original state radially expanded of maximum outer diameter of either the stabilizing section 130 or the sealing section 135; and (ii) less than or equal to the inner diameter of the lumen of the microcatheter through which the embolization device is deliverable to the target site. The narrow outer diameter of each of the respective proximal section 110 and intermediate section 115 may, but need not necessarily, be substantially equal. Regardless of the state of any other section of the embolization device at any given time, the proximal section 110 preferably is maintained at all times substantially unchanging in outer diameter. That is, preferably no radial constriction is required of the proximal section 110 during delivery through the lumen of the microcatheter to the target site nor does the proximal section 110 undergo any radial expansion upon exiting from the distal end/tip of the microcatheter. During manufacture the proximal section may be pre-formed (e.g., heat set) as a radially non-transitioning (i.e., not radially self-expanding) region having the aforementioned narrow outer diameter.

Conventional tubular braided implantable endovascular embolization devices while being delivered through the microcatheter to the target site (e.g., aneurysm) may become twisted about a longitudinal/axial axis. When deployed at the target site, if the conventional tubular braided embolization device is twisted the efficiency of diversion or disruption of blood flow away from the aneurysm or through blood vessel may be compromised. To prevent or minimize undesirable twisting during delivery and hence ensure maximum efficiency of diversion of blood flow, the tubular braided implantable endovascular embolization device 100 in accordance with the present disclosure is preferably independently freely rotatable 360 degrees relative to the delivery wire 120. Therefore, during advancement in a distal direction through the microcatheter, the free rotation of the tubular braided implantable endovascular embolization device 100 relative to the delivery wire 120 prevents or minimizes twisting in the axial/longitudinal direction and if minimum twisting occurs allows for automatic self-untwisting, that otherwise may compromise efficiency of diversion of blood flow. FIG. 1E illustrates an enlarged section 1E in FIG. 1A of an example independently freely rotatable connection of the tubular braided implantable endovascular embolization device 100 relative to the delivery wire 120. The independently freely rotatable connection includes an enlarged distal end 145a (e.g., ball or other distal interference member) disposed within the internal cavity of the sealing section 135 and having a maximum outer diameter greater than the inner diameter of the inner channel 140 of the proximal section 110 preventing passage or escape therethrough. A tether 145b extending through the inner channel 140 of the proximal section 110 of the tubular braided implantable endovascular embolization device 100 connects the enlarged distal end 145a to a distal end of the delivery wire 120. The tether 145b preferably has an outer diameter sized to provide sufficient clearance space when threaded through the inner channel 140 of the proximal section 110 allowing independent free 360 degrees rotation of the embolization device 100 relative to the delivery wire 120. In addition, during detachment the reduced outer diameter of the tether 145b relative to that of the delivery wire 120 hastens the time for severing (e.g., thermal heating, mechanical, or otherwise) releasing the delivery wire 120 from the embolization device 100 remaining implanted at the target site (e.g., in the aneurysm). The enlarged distal end 145a and severed portion of the tether 145b attached thereto remains in place within the implanted embolization device 100 while the delivery wire 120 and microcatheter 125, either independently in series one after the other or simultaneously together, are withdrawn from the body.

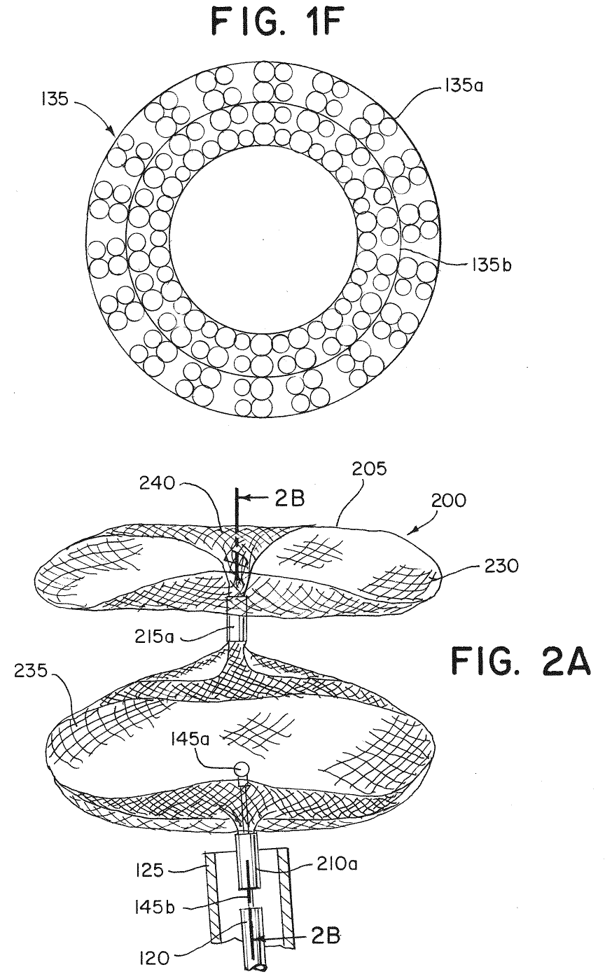

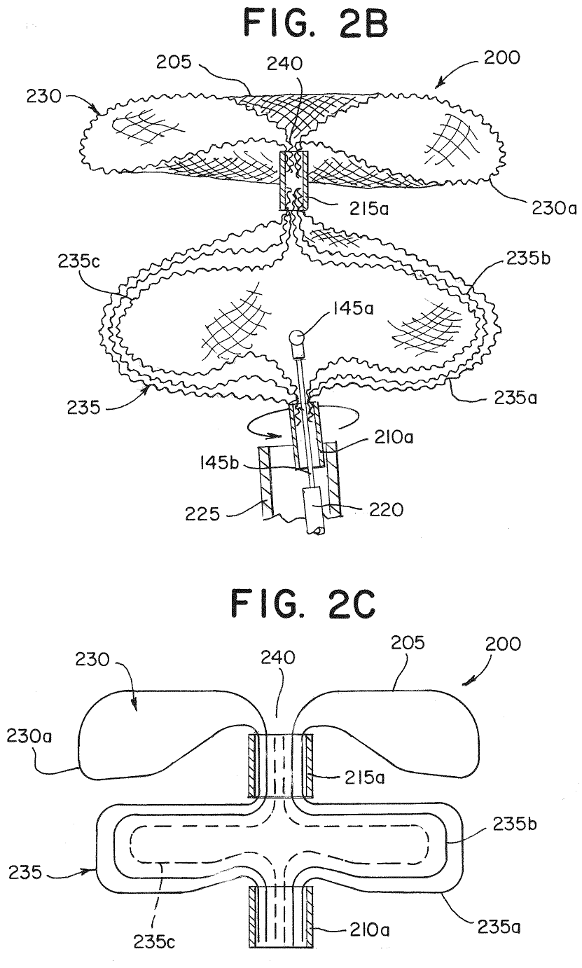

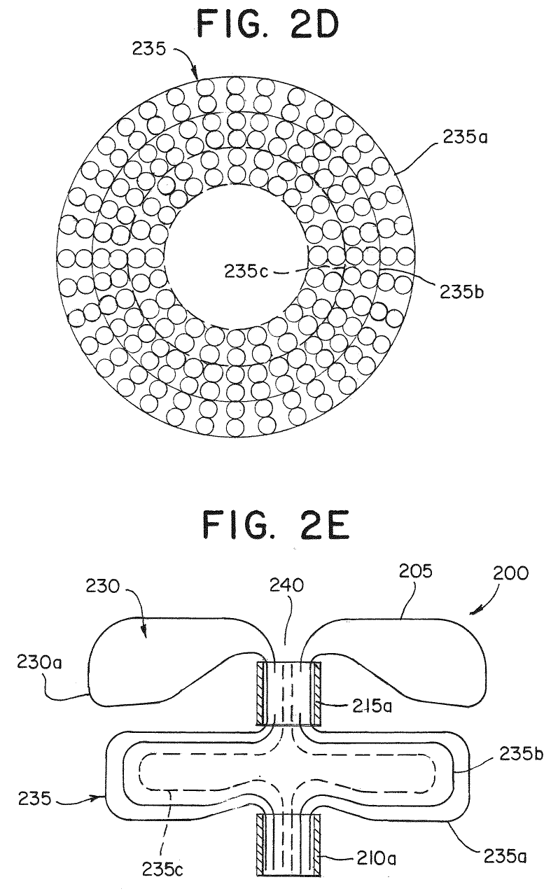

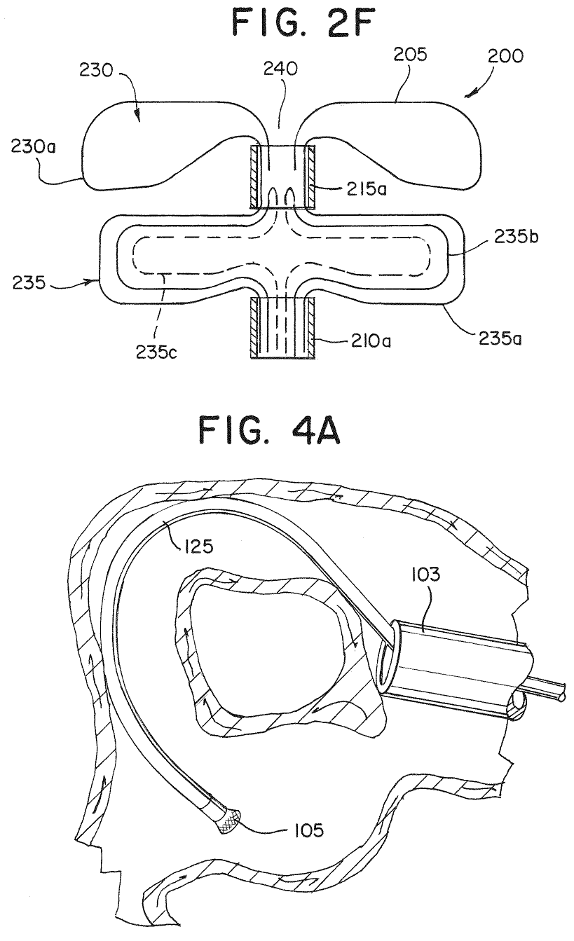

The description above refers to the example depicted in FIGS. 1A-1E of the tubular braided implantable endovascular embolization device 100 with a double layer sealing section 135 comprising two braided layers 135a, 135b, nested one inside the other. In alternative examples of the tubular braided implantable endovascular embolization device 200 shown in FIGS. 2A-2F the sealing section 235 comprises three braided layers 235a, 235b, 235c (i.e., triple layer) providing greater stiffness and enhanced diversion or disruption of blood flow compared to the double layer sealing section 135 in FIGS. 1A-1E having only two nested braided layers 135a, 135b. Regardless of the number of nested multi-layers (e.g., double layer or triple layer) comprising the multi-layer sealing section, the folded over tubular braid forms the only single layer of the stabilizing section and at least one layer of the multi-layer sealing section. Addressing the multi-layer sealing section (e.g., two braided layers or three braided layers), the folded over tubular braid (forming the folded over atraumatic distal surface) may represent: (i) only a single braided layer; or (ii) two braided layers. If respective free edges of the folded over tubular braid are substantially aligned with one another together representing a proximal end (opposite that of the folded over atraumatic distal surface) then the folded over tubular braid forms two layers of the multi-layer sealing section. Examples in which the folded over tubular braid forms two braided layers of the multi-layer sealing section are shown in FIG. 1D (double layer sealing section 135 wherein both braided layers 135a, 135b are formed by the folded over tubular braid) and FIG. 2C (triple layer sealing section 235 including two braided layers 235a, 235b formed by the folded over tubular braid and a separate supplemental structural obstructing layer (e.g., third braided layer 235c)). Otherwise, the respective free edges of the folded over tubular braid may be offset in the longitudinal/axial direction from one another. In which case, the folded over tubular braid forms only a single braided layer of the multi-layer sealing section along with at least two supplemental structural obstructing layers (e.g., supplemental tubular braid(s), coil(s), and/or suture(s)) separate from that of the folded over tubular braid. Examples in which the folded over tubular braid forms only a single braided layer of the multi-layer sealing section are shown in FIG. 1C (double layer sealing section 135 formed by two braided layers 135a, 135b) and FIGS. 2E & 2F (depicting different examples of triple layer sealing section 235 formed by three braided layers 235a, 235b, 235c). Specifically, in FIG. 1C the folded over tubular braid forms only a single braided layer (e.g., outer braided layer 135a) of the double layer sealing section 135 along with a separate supplemental structural obstructing layer (e.g., inner braided layer 135b). While in the triple layer sealing section 235 example in FIG. 2E the folded over tubular braid forms only a single braided layer (e.g., outer braided layer 235a) together with two supplemental structural obstructing layers (e.g., a first supplemental structural obstructing non-folded tubular braided layer 235b and a second supplemental structural obstructing non-folded tubular braided layer 235c) separate from each other and from the outer braided layer 235a. In FIG. 2F of yet another example of the triple layer sealing section the folded over tubular braid forms only a single braided layer (e.g., outer braided layer 235a) and separate therefrom two supplemental structural obstructing layers (e.g., braided layers 235b, 235c represented by a supplemental structural obstructing tubular braid folded over itself with opposing free edges substantially aligned in the same direction).

Regardless of the arrangement (e.g., substantially aligned or offset in a longitudinal/axial direction), each free edge of all respective layers (e.g., the folded over tubular braid and/or supplemental structural obstructing layer(s)) are secured in place via a securement mechanism along one of the intermediate section 115, 215, the sealing section 135, 235 or the proximal section 110, 210, but preferably not along the stabilizing section 130, 230 to minimize stiffness while maximizing compressibility and positioning. Free edges of the respective layers comprising the embolization device may be secured using a mechanical securement device (e.g., marker band), suture(s), adhesive, and/or welded, etc. In the illustrative examples in FIGS. 1C & 2C, 2E & 2F each free edge of the respective layers forming the embolization device 100, 200 is secured at either the intermediate section 115, 215 or the proximal section 110, 210 via an intermediate marker band 115a, 215a and a proximal marker band 110a, 210a, respectively. Whereas in FIG. 1D, no free edge of any layer of the embolization device coincides with the intermediate section 115 thereby eliminating the need for an intermediate marker band, employing only a proximal marker band 110a. The marker band (e.g., intermediate or proximal) may be crimped, compressed, or physically deformed around the respective section (e.g., intermediate or proximal) of the embolization device securing in place the free edges of the layers of the embolization device. Alternative methods for securing in place the free edges of the layers of the embolization device include welding and/or adhesive. While another contemplated method of securement of the free edges of the layers of the embolization device is via suture(s) made of a synthetic polymer or natural polymer (e.g., collagen). The securement mechanism for each free edge need not be the same and multiple securement methods may be employed. Regardless, of the securement mechanism used, the free edge of any layer of the embolization device is preferably not secured within the stabilizing section 130, 230 thereby minimizing stiffness while optimizing compressibility and positioning.

Use of the intermediate marker band 115a is described above secured (e.g., crimped, deformed, or compressed) about an intermediate section 115 that is pre-formed to be radially non-transitioning (i.e., not radially self-expanding) of reduced outer diameter relative to that of the stabilizing section securing in place free edges of the layers comprising the embolization device. As mentioned earlier, alternatively the intermediate section 115 may be pre-formed (e.g., heat set) to be radially self-expanding and the intermediate marker band 115a freely slidable in a longitudinal/axial direction to a desired position along the intermediate section 115 while in a radially constrained state. During deployment of the embolization device at the target site, as the stabilizing section 130 emerges/exits from the distal end/tip of the microcatheter and self-expands radially the freely slidable intermediate marker band 115a is automatically displaced in a proximal direction simultaneously self-adjusting in size the respective stabilizing section 130 and sealing section 135. As the intermediate marker band 115a is displaced towards the proximal section, the stabilizing section 130 while radially self-expanding increases in size and as a result the sealing section 135 simultaneously decreases in size. Accordingly, based on its position the freely slidable intermediate marker band automatically self-adjusts in size the respective stabilizing section 130 and the sealing section 135 dependent on the anatomy of the aneurysm. In such example since the intermediate marker band is freely slidable in the longitudinal/axial direction between the stabilizing and sealing sections, the free edges of the respective layers comprising the embolization device may be secured in place via an alternative method (e.g., adhesive, welding, and/or at least one suture).

The outer diameter of the multi-layer sealing section preferably remains substantially unchanged despite the addition of more than one supplemental structural obstructing layer so that the same size microcatheter may be employed for delivery to the target site regardless of whether the sealing section is double layer or triple layer. Implementation of this goal is illustrated in the exemplary double and triple layer sealing sections of different embolization devices depicted in FIGS. 1F & 2D, respectively, represented in a fully radially constricted state (i.e., the series of nested multi-layers comprising the sealing section being in direct physical contact with one another having a minimum outer diameter) deliverable through an example microcatheter having an inner diameter of approximately 0.017″. Despite the triple braided layer sealing section 235 including an additional layer in the example of FIG. 2D the outer diameter (approximately 0.0048″) remains substantially the same (e.g., de minimis reduction) as that of the outer diameter (approximately 0.0049″) of the double braided layer sealing section 135 example of FIG. 1F such that either example embolization device is deliverable to the target site using the same size microcatheter with the lumen having an inner diameter of approximately 0.017″.

Each of the double layer and triple layer sealing section examples in FIGS. 1F & 2D, respectively is discussed in further detail. Referring to the double layer sealing section 135 example depicted in a radially constricted state in FIG. 1F, the three concentric circles denoted by the thicker lines represent boundaries of the two braided layers (e.g., a first braided layer 135a disposed between the two outermost thicker line circles and a second braided layer 135b disposed between the two innermost thicker line circles). That space disposed radially inward of the innermost thicker line circle representing the inner channel 140. Each of the two braided layers 135a, 135b includes a total of 72 wires (e.g., 36 wires with each wire having an outer diameter approximately 0.001″ and 36 wires with each wire having an outer diameter approximately 0.0008″) woven into a desired pattern (e.g., diamond pattern). While in a radially constricted state with the respective two braided layers 135a, 135b in direct physical contact with one another (as depicted in FIG. 1F) the sealing section 135 has a maximum outer diameter of approximately 0.0049″ deliverable to a target site through the lumen of a microcatheter having an inner diameter approximately 0.017″. In this dual layer sealing section 135 example, the inner channel 140 has an inner diameter of approximately 0.009″.

Referring to the triple layer sealing section 235 example depicted in a radially constricted state in FIG. 2D, the four concentric circles denoted by the thicker lines represent boundaries of the respective three braided layers (e.g., first braided layer 235a disposed between the two outermost thicker line circles, a second braided layer 235b disposed between the two intermediate thicker line circles, and a third braided layer 235c disposed between the two innermost thicker line circles). Each of the first and second braided layers 235a, 235b have 72 wires with each wire having an outer diameter of approximately 0.0008″, while the third braided layer 235c has 48 wires with each wire having an outer diameter of approximately 0.0008″. Therefore, among the three braided layers, there are a total of 192 wires with each wire having an outer diameter approximately 0.0008″. While in a radially constricted state with the respective triple layers 235a, 235b, 235c in direct physical contact with one another (as depicted in FIG. 2D) the sealing section 235 has a maximum outer diameter of approximately 0.0048″ deliverable to a target site through the lumen of a microcatheter having an inner diameter approximately 0.017″. In this triple layer sealing section 235 example, the additional third braided layer 235c results in a de minimis reduction of the inner diameter of the inner channel 240 to approximately 0.008″ in comparison to the inner diameter of approximately 0.009″ of the inner channel 140 of the double braid layer sealing section in FIG. 1F.

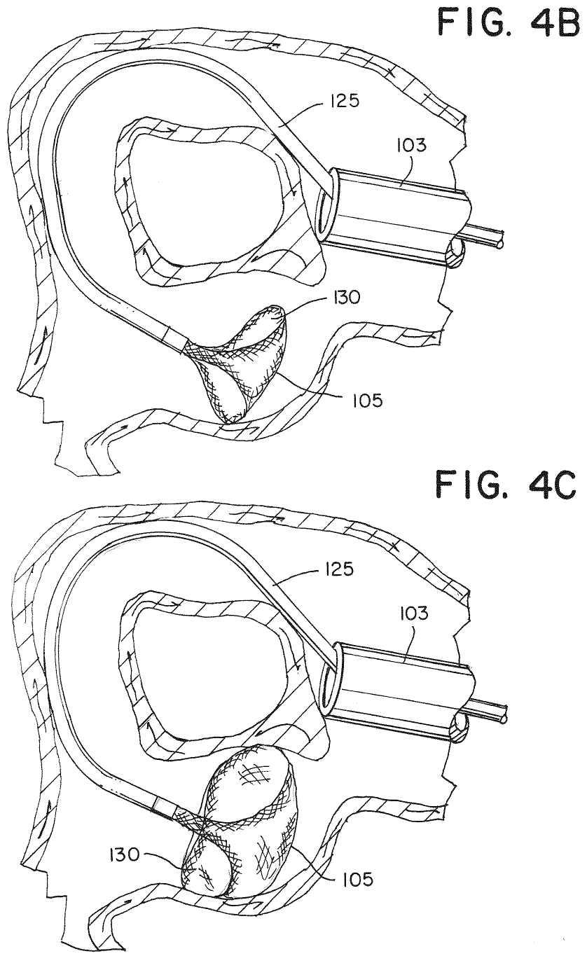

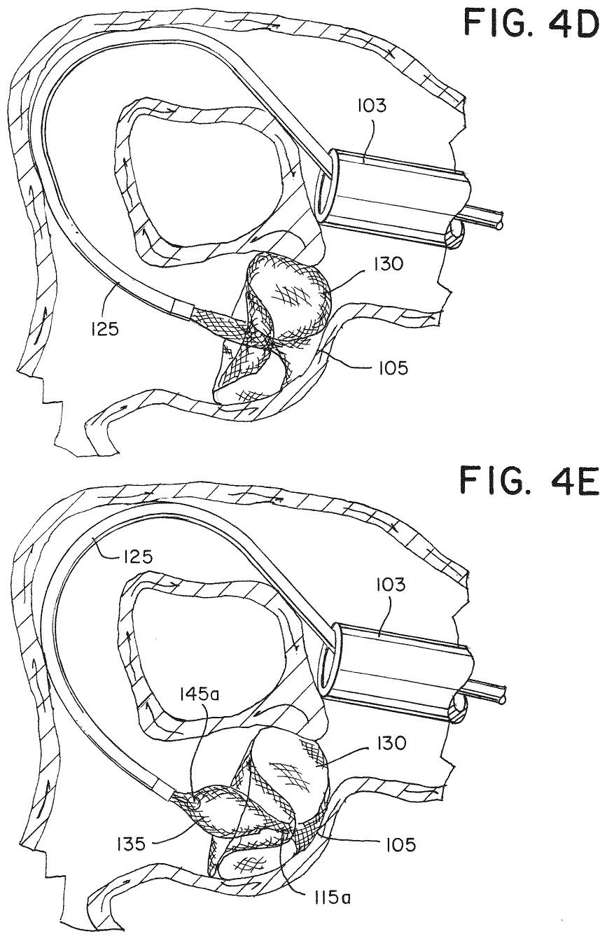

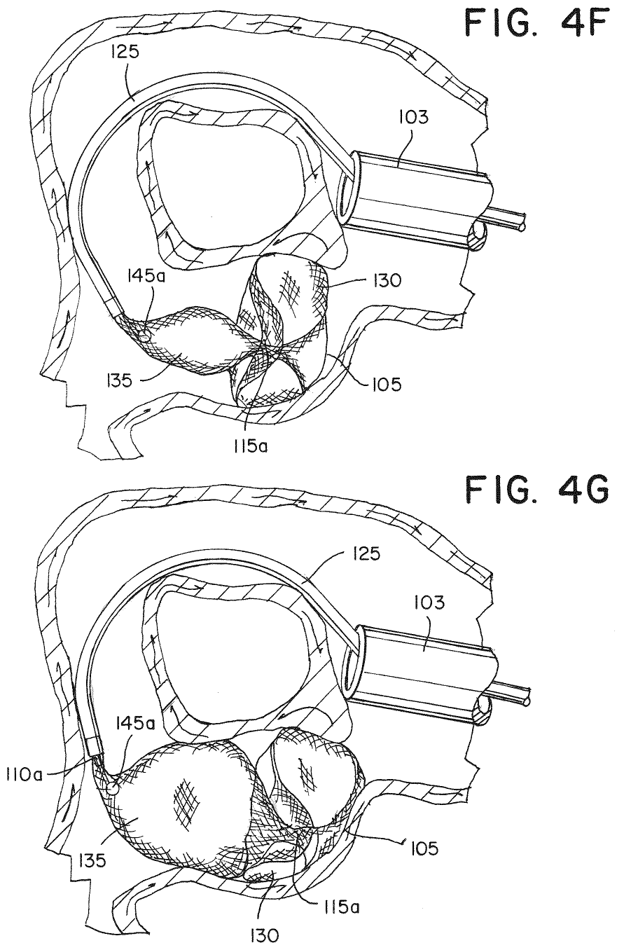

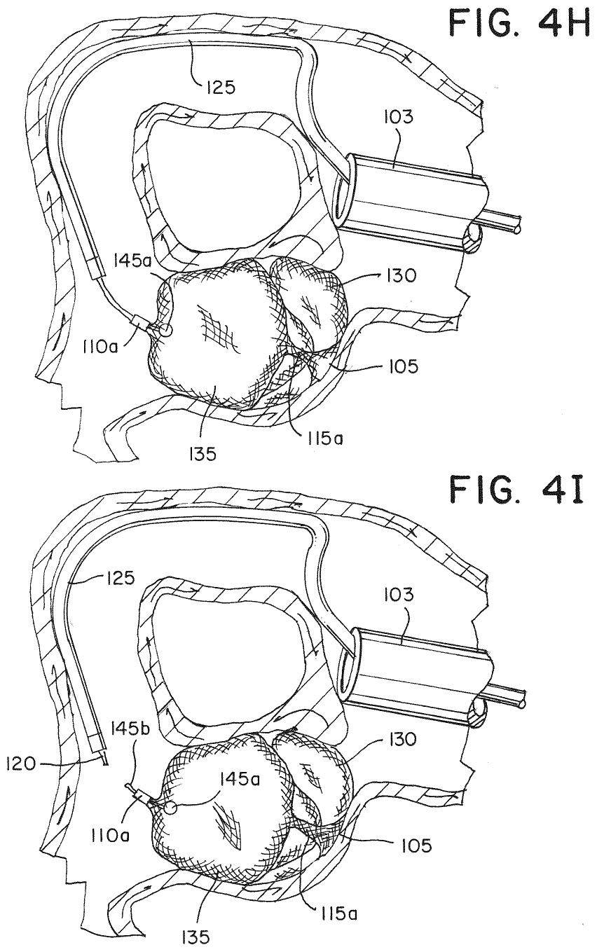

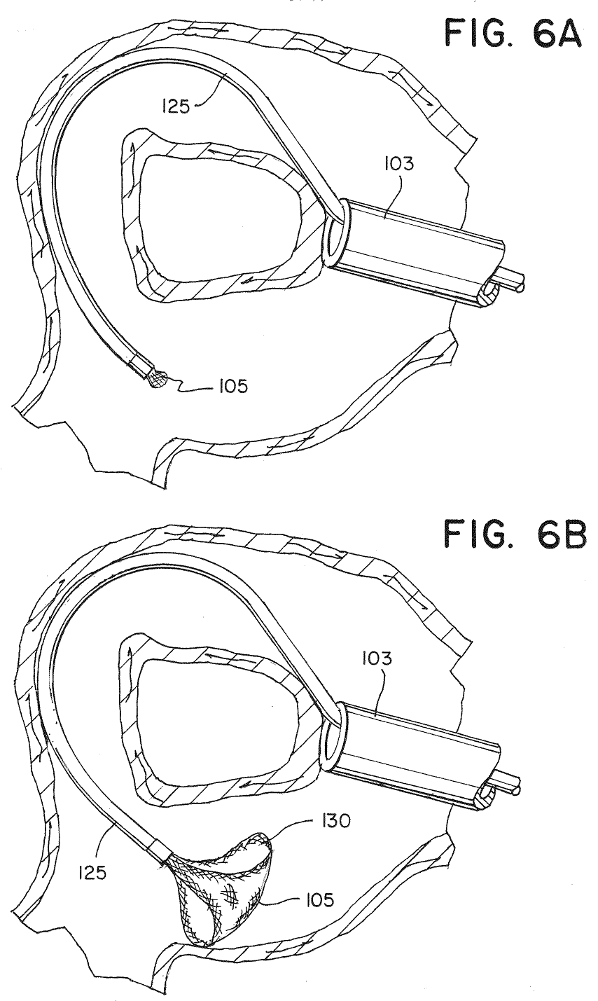

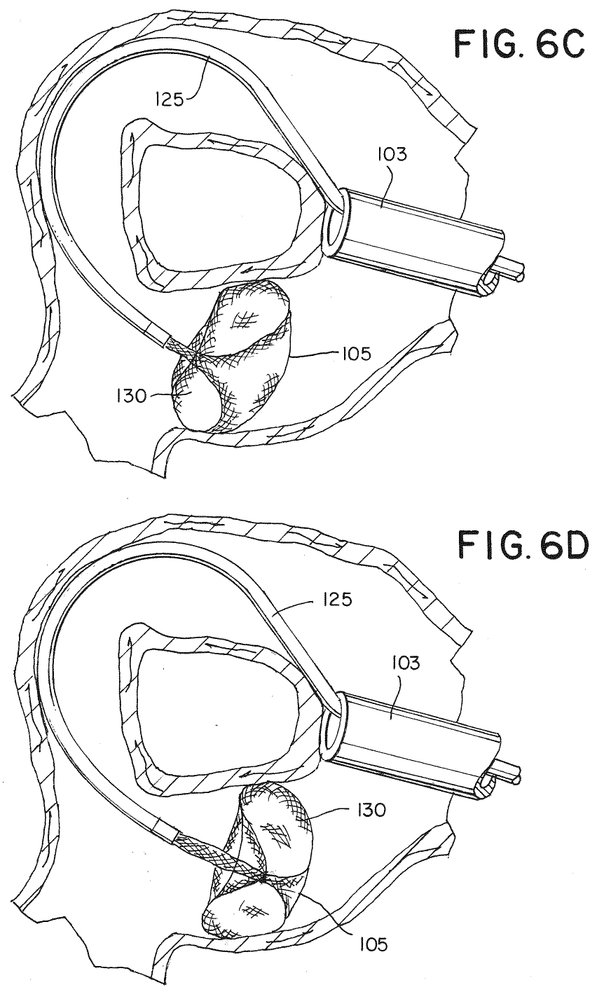

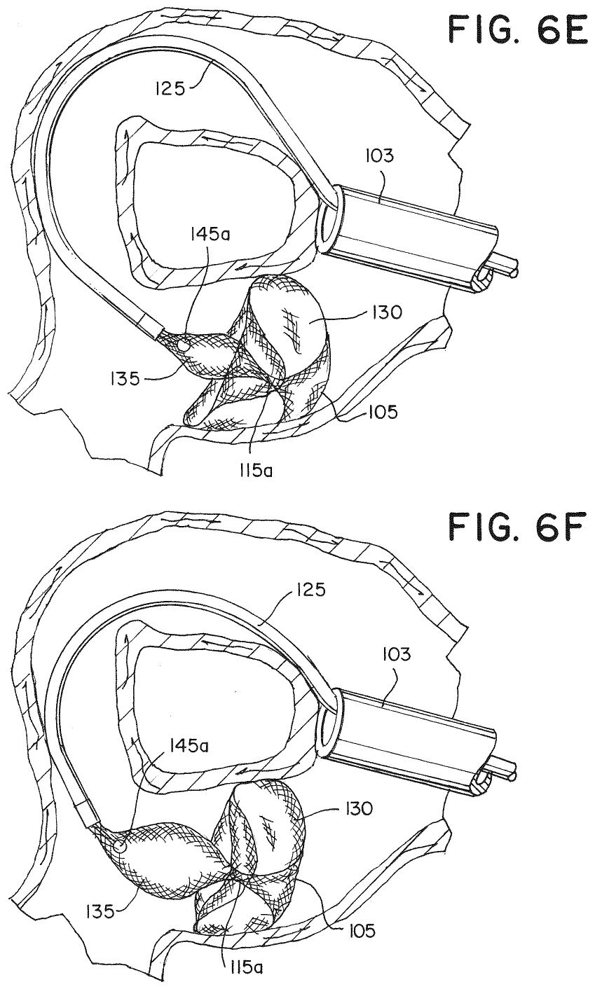

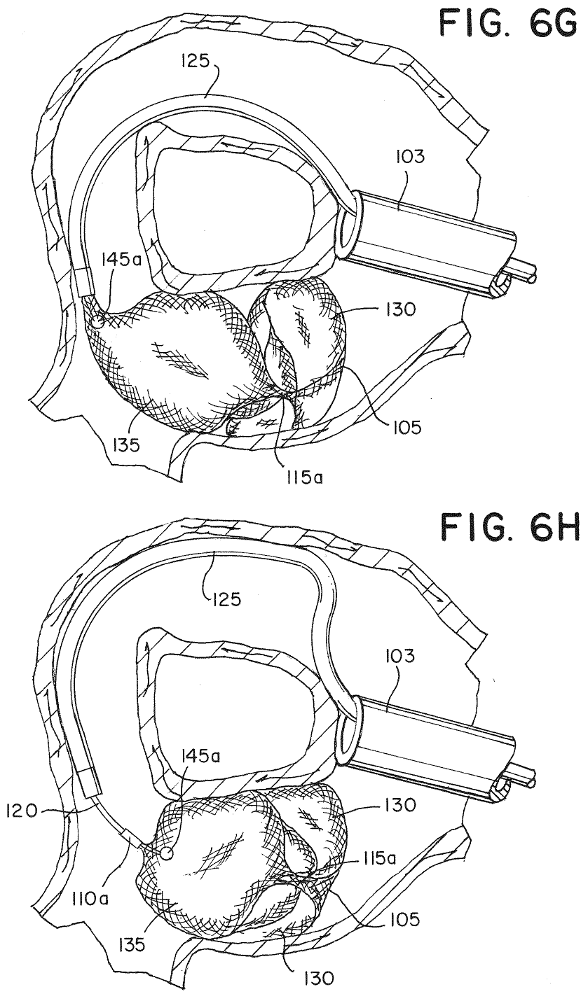

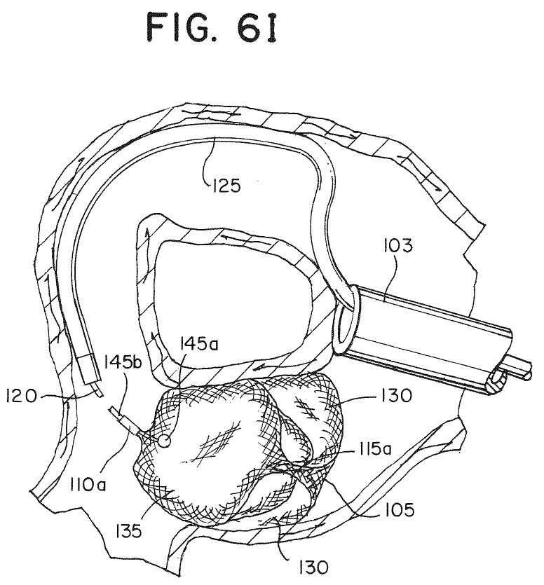

FIGS. 4A-4I depict sequential stages during delivery through the microcatheter 125 and deployment of the tubular braided implantable endovascular embolization device 100 having a double layer sealing section (as in the example of FIGS. 1A-1F) in treatment of an aneurysm. It is noted that the tubular braided implantable endovascular embolization device, regardless of the number of layers comprising the multi-layer sealing section (e.g., double layer or triple layer), undergoes the same sequential stages of delivery and deployment depicted in FIGS. 4A-4I. By way of illustrative example, FIGS. 4A-4I depict the delivery and deployment of the tubular braided implantable endovascular embolization device 100 in the treatment of an aneurysm, while FIGS. 6A-6I depict sequential stages of delivery and deployment of the tubular implantable endovascular embolization device 100 having a double layer sealing section (as in the example in FIGS. 1A-1F) in the restricting or impeding of blood flow in a vessel (e.g., a vessel in the brain or peripheral vasculature). Initially, not depicted in FIGS. 4A-4I, a guide wire and guide catheter 103 are navigated, either independently in series or simultaneously together, through the vasculature to the target site. The guidewire is then withdrawn in a proximal direction while the guide catheter 103 remains in place within the vessel. Alternatively, the guide catheter 103 may be eliminated altogether wherein the guide wire and microcatheter 125 are navigated, either independently in series or simultaneously together, through the vasculature. Referring to FIG. 4A the microcatheter 125 is advanced through the guide catheter 103 and out from its distal most end/tip at a proximal face or side of the aneurysm. Next, in a radially constricted state the tubular braided implantable endovascular embolization device 100 while radially constrained within the lumen of the microcatheter 125 is advanced (i.e., pushed) in a distal direction using the delivery wire 120 secured thereto. While in the radially constricted state, the stabilizing section 130 and sealing section 135 are reduced in outer diameter smaller than or equal to the inner diameter of the lumen of the microcatheter 125. Each of the intermediate section 115 and the proximal section 110 has a narrow outer diameter that is: (i) smaller relative to that of the natural, default, original state radially expanded of maximum outer diameter of either the stabilizing section 130 or the sealing section 135; and (ii) less than or equal to the inner diameter of the lumen of the microcatheter 125 through which the embolization device is deliverable to the target site. Thus, no further radial constriction (i.e., no further reduction in outer diameter) is imposed on the intermediate section 115 and proximal section 110 of the embolization device when introduced and advanced through the lumen of the microcatheter 125 and hence undergo no radial expansion upon exiting from the distal end/tip thereof. The first to emerge from the distal end/tip of the microcatheter 125 is the folded over atraumatic distal surface 105 of the embolization device (FIG. 4A) thereby preventing or minimizing risk of damage to the wall of the sac/dome of the aneurysm. With continued advancement (i.e., pushing) in a distal direction of the delivery wire 120, upon exiting from the distal end/tip of the microcatheter 125 the stabilizing section 130 automatically self-expands radially to an implanted shape and size dependent on the anatomy (e.g., shape and size) of the aneurysm in which it is implanted (depicting partial radial expansion as the sealing section 135 partially emerges from the microcatheter 125 in FIG. 4B). FIG. 4C depicts the stabilizing section 130 fully exited from the distal end/tip of the microcatheter 125 with regions of the surface of the stabilizing section 130 being in direct physical contact imposing a force against the wall of the sac/dome of the aneurysm. Additional advancement (i.e., pushing) of the delivery wire 120 assisted by the heightened stiffness of the intermediate section 115 advances the stabilizing section 130 further distally in the sac/dome of the aneurysm maximizing direct physical contact (i.e., coverage) of the folded over atraumatic distalmost surface 105 with the wall of the sac/dome of the aneurysm (FIG. 4D). Maximized coverage of the aneurysm wall of the sac/dome provided by the stabilizing section 130 is particularly advantageous if the aneurysm is ruptured. Following the intermediate section 115 at in the example of FIG. 1A doesn't undergo radial expansion upon exiting from the distal end/tip of the microcatheter 125, the sealing section 135 is next to emerge self-expanding radially to its implanted state dependent on the anatomy (e.g., shape and size) of the aneurysm in which it is implanted (FIGS. 4E-4G) while spanning the entrance (i.e., neck). The sealing section 135 while implanted (i.e., deployed or radially self-expanded) is repositionable to be properly seated at the neck of the aneurysm to maximize diversion or disruption of blood flow. In this regard, the intermediate section 115 (narrower in outer diameter and stiffer (i.e., more rigid) relative to that of the stabilizing section 130 while in a radially expanded implanted state) acts like a hinge allowing independent and angular offset positioning of the sealing section 135 relative to that of the stabilizing section 130 anchored in place. While visible under imaging (e.g., fluoroscopic imagery), proper positioning of the sealing section 135 at the neck of the aneurysm is realized by the physician or interventionalist manipulating (e.g., partially retracting/withdrawing in a proximal direction and/or advancing in a distal direction) the delivery wire 120. While repositioning the sealing section 135 at the neck of the aneurysm during advancement in the distal direction of the delivery wire 120 the distalmost folded over atraumatic distal surface 105 of the embolization device advantageously serves as a bumper preventing damage to the wall of the sac/dome of the aneurysm. FIG. 4H depicts the proximal section 110 of the implanted embolization device fully exited from the distal end/tip of the microcatheter with the sealing section 135 properly seated at the neck of the aneurysm. When properly seated at the neck of the aneurysm, as illustrated in FIG. 4H, preferably only a minimal portion (e.g., ≤approximately 5%) of the sealing section 135 protrudes or extends into the adjacent parent vessel, i.e., substantially all (e.g., ≥approximately 95%) of the sealing section 135 is disposed within the aneurysm (i.e., neck or sac/dome). Lastly, detachment takes place by severing (e.g., via electrolytic and/or mechanical detachment such as a pull wire) the tether 145b thereby releasing it from the delivery wire 120. The delivery wire 120 and microcatheter 125, either independently in series one following the other or simultaneously together, are withdrawn in a proximal direction from the body. Detached from the delivery wire 120, the embolization device 100 (together with the enlarged distal end 145a and severed portion of the tether 145b) remains implanted in the aneurysm (FIG. 4I). Alternative delivery systems and detachment mechanisms are contemplated and within the scope of the present invention.

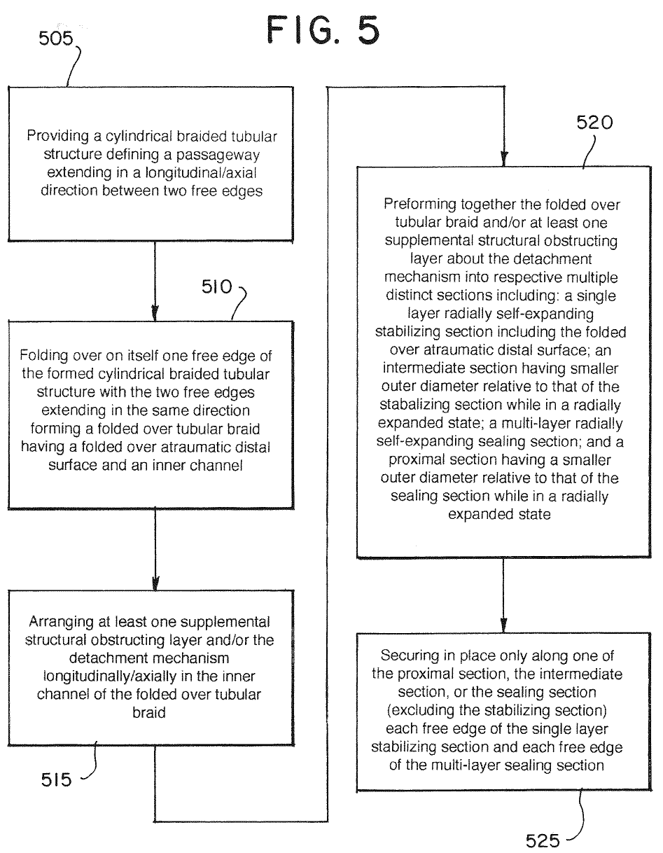

The exemplary flow chart in FIG. 5 depicts the steps undertaken during the manufacture of the tubular braided implantable endovascular embolization device in accordance with the present invention. Initially, a cylindrical tubular braided structure is formed/provided in step 505 using a plurality of wires (each wire preferably having an outer diameter of ≤approximately 0.001″) woven into a desired pattern (e.g., full diamond pattern). The cylindrical tubular braid defines a passageway extending in a longitudinal/axial direction between opposing free edges at either end. In step 510 one free edge of the formed cylindrical tubular braided structure is folded over itself and pulled in the same direction as the other free edge to form the folded over tubular braid having a folded over atraumatic distal surface. The respective free edges of the folded over tubular braid may be either substantially aligned with one another (together forming the proximal end opposite the folded over atraumatic distal surface) or offset from one another in the longitudinal/axial direction. Prior to preforming the multiple distinct sections of the embolization device, in step 515 one or more supplemental structural obstructing layer (e.g., supplemental structural obstructing tubular braid, coil(s), or suture(s)) forming the multi-layer sealing section and/or the detachment mechanism (e.g., enlarged distal end 145a and tether 145b attached thereto) is longitudinally/axially arranged in the inner channel of the folded over tubular braid. In the particular detachment mechanism depicted in FIG. 1E, the enlarged distal ball 145a is aligned with the to be formed sealing section 135 while the tether 145b extends through the to be formed proximal section 110 secured at its proximal end to the distal end of the delivery wire 120. Next in step 520, around the properly positioned detachment mechanism the folded over tubular braid and/or supplemental structural obstructing layer(s) are pre-formed (e.g., heat set) into the multiple distinct sections: stabilizing section, intermediate section, sealing section, and proximal section. Each of the stabilizing and sealing sections are preformed to be radially self-expanding (i.e., radially collapsible or constricted during delivery to the target site through the microcatheter, and upon exiting from the distal end/tip of the microcatheter is radially self-expandable radially to an implanted size and shape conforming to the anatomy (e.g., size and shape) of the sac/dome of the aneurysm). The stabilizing and sealing sections may, but need not necessarily, have the same maximum outer diameter while in a natural, default, or original state (e.g., radially expanded state). Intermediate section 115 and proximal section 110 each have a narrow outer diameter that is: (i) smaller relative to that of the natural, default, original state radially expanded of maximum outer diameter of either the stabilizing section 130 or the sealing section 135; and (ii) less than or equal to the inner diameter of the lumen of the microcatheter through which the embolization device is deliverable to the target site. Regardless of the state of any other section of the embolization device at any given time, the proximal section 110 preferably is maintained at all times substantially unchanging in outer diameter. Each respective free edge of the layers comprising the embolization device (e.g., folded over tubular braid and/or the one or more supplemental structural obstructing layers) is then secured in place along one of the intermediate section 115, the sealing section 130, or the proximal section 110 (preferably excluding or free from the stabilizing section) in step 525. Securement mechanisms to maintain in position free edges of the respective layers of the embolization device include: mechanical devices (e.g., crimped marker band, suture(s), welding, and/or adhesive. The completed/assembled folded over tubular implantable endovascular embolization device has a predetermined length L (e.g., approximately 1″) from the folded over atraumatic distal surface 105 to the opposite proximal end (not including the tether 145b extending therefrom or delivery wire attached thereto) selected, as desired, based on the anatomy (e.g., size and/or shape) of the aneurysm being treated.

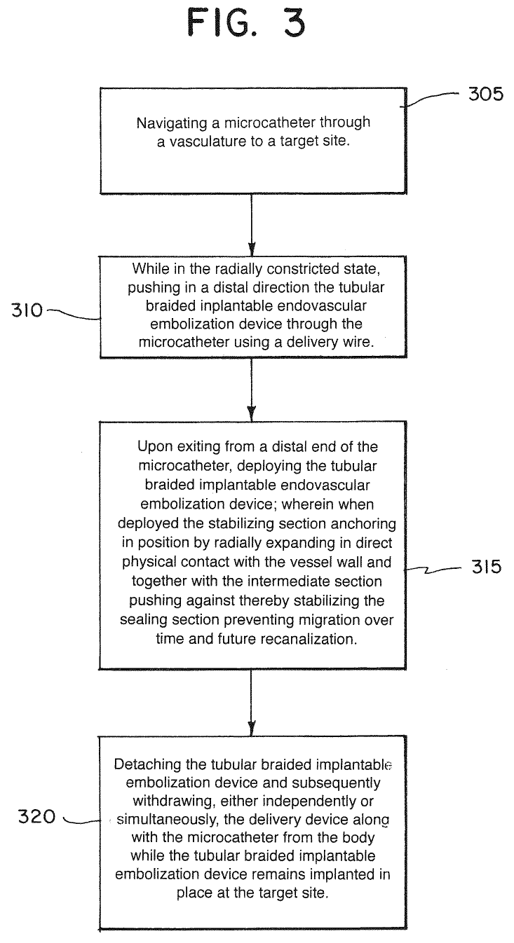

Referring to the exemplary flow chart of FIG. 3, in operation initially in step 305 the microcatheter 125 is navigated through a vasculature to the target site. This step may be accomplished in several different ways depending on the catheter delivery system used. In one example a guide wire and microcatheter, independently in series one after the other or simultaneously together, are navigated through the vasculature to the target site (e.g., on a proximal side of the aneurysm or blood vessel (e.g., in the brain or periphery vessel)) followed by subsequent removal of the guide wire leaving the microcatheter 125 in place. Alternatively, a guide catheter 103 along with the guide wire, either independently in series one after the other or simultaneously together, are navigated through the vasculature with subsequent withdraw of the guide wire prior to advancement of the microcatheter 125 emerging from the distal end/tip of the guide catheter 103. While in the radially constricted state, in step 310 the tubular braided implantable endovascular aneurysm embolization device 100, 200 is advanced (e.g., pushed) in a distal direction through the microcatheter 125 using the delivery wire 120. In step 315, upon exiting from a distal end/tip of the microcatheter 125 the tubular braided implantable endovascular aneurysm embolization device 100, 200 is deployed at the target site (e.g., in the aneurysm or blood vessel). First to emerge or exit from the distal end/tip of the delivery device (e.g., microcatheter 125) is the stabilizing section 130, 230 self-expanding radially to an implanted state dependent on the anatomy of the target site (e.g., aneurysm or blood vessel). In a fully deployed state the stabilizing section 130, 230 is in direct physical contact imposing a force against the vessel wall (e.g., sac/dome wall of the aneurysm) stabilizing, anchoring, or maintaining its position therein. Next to emerge from the distal end/tip of the delivery device (e.g., microcatheter 125) without undergoing radial expansion the intermediate section 115, 215 provides column strength between while also acting as a hinged connection point allowing independent positioning between the stabilizing section 130, 230 and the sealing section 135, 235. Upon exiting from the distal end/tip of the delivery device (e.g., microcatheter 125), the sealing section 135, 235 radially self-expands to span the neck of the aneurysm while its multiple nested layers divert, disrupt, or impede blood flow therethrough to the target site (e.g., aneurysm or blood vessel). In the treatment of an aneurysm, the multi-layer sealing section 135, 235 provides sufficient, but not complete/total/full, diversion or disruption of blood flow therethrough to optimize healing at the neck of the aneurysm. The stabilizing section 130, 230 while anchored in place together with the intermediate section 115, 215 (enhanced in stiffness) pushes the sealing section 135, 235 in the proximal direction towards thereby stabilizing at the neck minimizing risk of distal migration into the aneurysm over time (i.e., future recanalization). While implanted and subject to visible imagery (e.g., fluoroscopic imagery), if necessary, the physician or interventionalist physically may manipulate (e.g., partially pulling/retracting in a proximal direction and/or advancing in a distal direction) the delivery wire 120 to properly seat the sealing section 135, 235 at the target site (e.g., neck of the aneurysm or blood vessel) thereby maximizing diversion or occlusion of blood flow. Now with the implant properly positioned at the target site, in step 320 the detachment mechanism (e.g., tether 145b) is severed/released leaving in place the tubular braided implantable endovascular embolization device 100, 200 (along with the severed portion of the tether 145b and enlarge distal end 145a) while the delivery wire 120, 220 and microcatheter 125, 225 either independently or simultaneously, is withdrawn in a proximal direction from the body.

Aspects of the present disclosure are also provided by the following numbered Clauses:

Clause 1: An endovascular embolization system comprising a braided implantable device (100, 200) including: a folded over tubular braid forming a folded over atraumatic distal surface (105, 205) and an inner channel (115, 215); wherein the folded over tubular braid is preformed into multiple distinct sections including: a stabilizing section (130, 230) including the folded over atraumatic distal surface (105, 205); a sealing section (135, 235) disposed proximally of the stabilizing section (130, 230); an intermediate section (115, 215) interposed directly between the stabilizing section (130, 230) and the sealing section (135, 235); the intermediate section (115, 215) having a stiffness greater than that of the stabilizing section (130, 230); and a proximal section (110, 210) disposed proximally of the sealing section (135, 235); wherein each of the stabilizing section (130, 230) and the sealing section (135, 235) are self-expanding between a radially expanded state when free from an externally applied radial force and a radially constricted state when subject to the externally applied radial force; each of the stabilizing section (130, 230) and the sealing section (135, 235) while in the radially expanded state have a maximum outer diameter larger than an outer diameter of each of the intermediate section (115, 215) and the proximal section (110, 210); the stabilizing section (130, 230) comprises only a single braided layer (130a, 230a) while the sealing section (135, 235) comprises a plurality of nested layers (135a, 135b, 235a, 235b, 235c) providing enhanced stiffness greater than that of the stabilizing section (130, 230) while disrupting blood flow passable through open space regions defined by the plurality of nested layers; and wherein the folded over tubular braid forms the single braided layer (130a, 230a) of the stabilizing section (130, 230) and at least one of the plurality of nested layers (135a, 135b, 235a, 235b, 235c) of the sealing section (135, 235).

Clause 2: The system of Clause 1, wherein the folded over tubular braid forms two braided layers of the plurality of nested layers (135a, 135b, 235a, 235b) of the sealing section (135, 235).

Clause 3: The system of Clause 2, wherein one of the plurality of nested layers (235c) of the sealing section (235) is a supplemental structural obstructing layer (235c) separate from the two braided layers (235a, 235b) of the folded over tubular braid; the supplemental structural obstructing layer (235c) further disrupting the blood flow passable through the open space regions defined by the plurality of nested layers (235a, 235b, 235c) of the sealing section (235).

Clause 4: The system of Clause 1, wherein the plurality of nested layers (135a, 135b, 235a, 235b, 235c) of the sealing section (135, 235) comprises: (i) the folded over tubular braid forming only one braided layer (135a, 235a) of the plurality of nested layers of the sealing section (135, 235); and (ii) at least one supplemental structural obstructing layer (135b, 235b, 235c) separate from the only one braided layer (135a, 235a) formed by the folded over tubular braid further disrupting the blood flow passable through the open space regions defined by the plurality of nested layers (135a, 135b, 235a, 235b, 235c) of the sealing section (135, 235).

Clause 5: The system of Clause 4, wherein the at least one supplemental structural obstructing layer comprises two supplemental structural obstructing layers (235b, 235c) separate from one another and separate from the only one braided layer (235a) of the folded over tubular braid; the two supplemental structural obstructing layers (235b, 235c) further disrupting the blood flow passable through the open space regions defined by the plurality of nested layers (235a, 235b, 235c) of the sealing section (235). Clause 6: The system of any of Clauses 1 through 5, wherein each of the plurality of nested layers (135a, 135b, 235a, 235b, 235c) is a braid layer, at least one coil, and/or at least one suture.

Clause 7: The system of any of Clauses 1 through 6, wherein along one of the intermediate section (115, 215), the proximal section (110, 210), or the sealing section (135, 235) is secured each free edge of the single braided layer (130a, 230a) of the stabilizing section (130, 230) and each free edge of the plurality of nested layers (135a, 135b, 235a, 235b, 235c) of the sealing section (135, 235).

Clause 8: The system of any of Clauses 1 through 7, wherein each of the intermediate section and the proximal section are preformed to be radially non-transitionable having a substantially unchanging outer diameter.

Clause 9: The system of any of Clauses 1 through 8, wherein the intermediate section is radially self-expanding and radially constrained by an intermediate marker band (115a, 215a) disposed about and freely slidable in a longitudinal direction along the intermediate section (115, 215) to vary in size the respective stabilizing section (130, 230) and the sealing section (135, 235) depending on positioning in the longitudinal direction of the intermediate marker band (115a, 215a).

Clause 10: The system of any of Clauses 1 through 9, wherein the braided implantable device (100, 200) is independently freely rotatably relative to a delivery wire (120, 220).

Clause 11: The system of any of Clauses 1 through 10, wherein the intermediate section (115, 215) allows for angular offset positioning between the stabilizing section (130, 230) and the sealing section (135, 235).

Clause 12: A method for disrupting blood flow at a target site using an endovascular embolization system comprising a braided implantable device (100, 200) including: a folded over tubular braid forming a folded over atraumatic distal surface (105, 205) and an inner channel (140, 240); wherein the folded over tubular braid is preformed into multiple distinct sections including: a stabilizing section (130, 230) including the folded over atraumatic distal surface (105, 205); a sealing section (135, 235) disposed proximally of the stabilizing section (130, 230); an intermediate section (115, 215) interposed directly between the stabilizing section (130, 230) and the sealing section (135, 235); the intermediate section (115, 215) having a stiffness greater than that of the stabilizing section (130, 230); and a proximal section (110, 210) disposed proximally of the sealing section (135, 235); wherein each of the stabilizing section (130, 230) and the sealing section (135, 235) are self-expanding between a radially expanded state when free from an externally applied radial force and a radially constricted state when subject to the externally applied radial force; each of the stabilizing section (130, 230) and the sealing section (135, 235) while in the radially expanded state have a maximum outer diameter larger than an outer diameter of each of the intermediate section (115, 215) and the proximal section (110, 210); the stabilizing section (130, 230) comprises only a single braided layer (130a, 230a) while the sealing section (135, 235) comprises a plurality of nested layers (135a, 135b, 235a, 235b, 235c) providing enhanced stiffness greater than that of the stabilizing section (130, 230) while disrupting blood flow passable through open space regions defined by the plurality of nested layers; and wherein the folded over tubular braid forms the single braided layer (130a, 230a) of the stabilizing section (130, 230) and at least one of the plurality of nested layers (135a, 135b, 235a, 235b, 235c) of the sealing section (135, 235); the method comprising the steps of: navigating a microcatheter (125, 225) through a vasculature to the target site; while in the radially constricted state, pushing in a distal direction the braided implantable device (100, 200) through the microcatheter (125, 225) using a delivery wire (120, 220); and upon exiting from a distal end of the microcatheter (125, 225), deploying the braided implantable device (100, 200) at the target site; wherein when deployed the stabilizing section (130, 230) anchoring in position by radially expanding in direct physical contact with a vessel wall at the target site and together with the intermediate section (115, 215) pushing against and stabilizing in place at the target site the sealing section (135, 235) thereby minimizing risk of migration in a distal direction over time; the plurality of nested layers (135a, 135b, 235a, 235b, 235c) maximizing disruption of without preventing blood flow passable through the open space regions defined by the plurality of nested layers (135a, 135b, 235a, 235b, 235c) of the sealing section (135, 235) when deployed at the target site, while the stabilizing section (130, 230) having only the single braided layer (130a, 230c) maintains maximum compressibility and positioning when deployed at the target site.

Clause 13: The method of Clause 12, wherein the deploying step further comprises the step of each of the stabilizing section (130, 230) and the sealing section (135, 235) independently self-adjusting in size and shape dependent on anatomy at the target site.

Clause 14: The method of any of Clauses 12 through 13, wherein the deploying step further comprises the step of repositioning the sealing section (135, 235) to be properly positioned at the target site with the folded over atraumatic distal surface (105, 205) of the stabilizing section (130, 230) acting as a bumper preventing damage to the vessel wall at the target site.

Clause 15: The method of any of Clauses 12 through 14, wherein the pushing step further comprises the step of minimizing twisting in a longitudinal direction of the braided implantable device (100, 200) by being independently freely rotatable relative to the delivery wire (120).

Clause 16: The method of any of Clauses 12 through 15, wherein the deploying step further comprises the step of permitting, via the intermediate section (115, 215) acting as a hinge, angular offset positioning of the sealing section (135, 235) while the stabilizing section (130, 230) is maintained anchored in place at the target site.

Clause 17: The method of any of Clauses 12 through 16, the deploying step further comprises the step of simultaneously self-adjusting in size the stabilizing section (130, 230) and the sealing section (135, 235), respectively, based on positioning in a longitudinal direction of an intermediate marker band (115a) disposed about and freely slidable along the intermediate section (115, 215).

Clause 18: The method of any of Clauses 12 through 17, wherein along one of the intermediate section (115, 215), the proximal section (110, 210), or the sealing section (135, 235) is secured each free edge of the single braided layer (130a, 230a) of the stabilizing section (130, 230) and each free edge of the plurality of nested layers (135a, 135b, 235a, 235b, 235c) of the sealing section (135, 235).

Clause 19: The method of any of Clauses 12 through 18, wherein the plurality of nested layers (135a, 135b, 235, 235b, 235c) of the sealing section (135, 235) include at least one supplemental structural obstruction layer (235b, 235c) separate from the folded over tubular braid; wherein the at least one supplemental structural obstruction layer is a supplemental braid layer, at least one supplemental coil, or at least one supplemental suture.

Clause 20: A method for manufacture of a tubular braided implantable endovascular embolization device including: a folded over tubular braid forming a folded over atraumatic distal surface (105, 205) and an inner channel (140, 240); wherein the folded over tubular braid is preformed into multiple distinct sections including: a stabilizing section (130, 230) including the folded over atraumatic distal surface (105, 205); a sealing section (135, 235) disposed proximally of the stabilizing section (130, 230); an intermediate section (115, 215) interposed directly between the stabilizing section (130, 230) and the sealing section (135, 235); the intermediate section (115, 215) having a stiffness greater than that of the stabilizing section (130, 230); and a proximal section (110, 210) disposed proximally of the sealing section (135, 235); wherein each of the stabilizing section (130, 230) and the sealing section (135, 235) are self-expanding between a radially expanded state when free from an externally applied radial force and a radially constricted state when subject to the externally applied radial force; each of the stabilizing section (130, 230) and the sealing section (135, 235) while in the radially expanded state have a maximum outer diameter larger than an outer diameter of each of the intermediate section (115, 215) and the proximal section (110, 210); the stabilizing section (130, 230) comprises only a single braided layer (130a, 230a) while the sealing section (135, 235) comprises a plurality of nested layers (135a, 135b, 235a, 235b, 235c) providing enhanced stiffness greater than that of the stabilizing section (130, 230) while disrupting blood flow passable through open space regions defined by the plurality of nested layers; and wherein the folded over tubular braid forms the single braided layer (130a, 230a) of the stabilizing section (130, 230) and at least one of the plurality of nested layers (135a, 135b, 235a, 235b, 235c) of the sealing section (135, 235); the method comprising the steps of: providing a cylindrical braided tubular structure defining a passageway in a longitudinal direction between two free edges at opposite ends; folding over on itself the cylindrical tubular braided structure with the two free edges extending in a same direction to form the folded over tubular braid having the folded over atraumatic distal surface (105, 205) and the inner channel (140, 240); arranging at least one supplemental structural obstructing layer (135b, 235b, 235c) and/or the detachment mechanism (145a, 145b) longitudinally in the inner channel (140, 240) of the folded over tubular braid; preforming together the folded over tubular braid and/or at least one supplemental structural obstructing layer (135b, 235b, 235c) about the detachment mechanism (145a, 145b) into respective multiple distinct sections including: the stabilizing section (130, 230); the intermediate section (115, 215); the sealing section (135, 235); and the proximal section (110, 210); wherein the detachment mechanism is secured within the inner channel (140, 240) of the proximal section (110, 210); and securing in place only along one of the proximal section (110, 210), the intermediate section (115, 215), or the sealing section (135, 235) each free edge of the single braided layer (130a, 230a) of the stabilizing section (130, 230) and each free edge of the plurality of nested layers (135a, 135b, 235a, 235b, 235c) of the sealing section (135, 235).

The descriptions contained herein are examples and not intended in any way to limit the scope of the present disclosure. As described herein, the present disclosure contemplates many variations and modifications of the tubular braided implantable embolization device having a folded over atraumatic distal end for use in diverting/disrupting blood flow to an aneurysm or impeding/restricting blood flow in a vessel (e.g., in the brain or peripheral vasculature). Modifications and variations apparent to those having skilled in the pertinent art according to the teachings of this disclosure are intended to be within the scope of the claims which follow.

What is claimed is:

1. An endovascular embolization system comprising:
a braided implantable device comprising:
a folded over tubular braid forming a folded over atraumatic distal surface and an inner channel; wherein the folded over tubular braid is pre-formed into multiple distinct sections including:
a stabilizing section including the folded over atraumatic distal surface;
a sealing section disposed proximally of the stabilizing section;
an intermediate section interposed directly between the stabilizing section and the sealing section; the intermediate section having a stiffness greater than that of the stabilizing section; and
a proximal section disposed proximally of the sealing section;
wherein each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the intermediate section and the proximal section; the stabilizing section comprises only a single braided layer while the sealing section comprises a plurality of nested layers providing enhanced stiffness greater than that of the stabilizing section while disrupting blood flow passable through open space regions defined by the plurality of nested layers; and wherein the folded over tubular braid forms the single braided layer of the stabilizing section and at least one of the plurality of nested layers of the sealing section.

2. The system in accordance with claim 1, wherein the folded over tubular braid forms two braided layers of the plurality of nested layers of the sealing section.

3. The system in accordance with claim 2, wherein one of the plurality of nested layers of the sealing section is a supplemental structural obstructing layer separate from the two braided layers of the folded over tubular braid; the supplemental structural obstructing layer further disrupting the blood flow passable through the open space regions defined by the plurality of nested layers of the sealing section.

4. The system in accordance with claim 1, wherein the plurality of nested layers of the sealing section comprises: (i) the folded over tubular braid forming only one braided layer of the plurality of nested layers of the sealing section; and (ii) at least one supplemental structural obstructing layer separate from the only one braided layer formed by the folded over tubular braid further disrupting the blood flow passable through the open space regions defined by the plurality of nested layers of the sealing section.

5. The system in accordance with claim 4, wherein the at least one supplemental structural obstructing layer comprises two supplemental structural obstructing layers separate from one another and separate from the only one braided layer of the folded over tubular braid; the two supplemental structural obstructing layers further disrupting the blood flow passable through the open space regions defined by the plurality of nested layers of the sealing section.

6. The system in accordance with claim 1, wherein each of the plurality of nested layers is a braid layer, at least one coil, and/or at least one suture.

7. The system in accordance with claim 1, wherein along one of the intermediate section, the proximal section, or the sealing section is secured each free edge of the single braided layer of the stabilizing section and each free edge of the plurality of nested layers of the sealing section.

8. The system in accordance with claim 1, wherein each of the intermediate section and the proximal section are preformed to be radially non-transitionable having a substantially unchanging outer diameter.

9. The system in accordance with claim 1, wherein the intermediate section is radially self-expanding and radially constrained by an intermediate marker band disposed about and freely slidable in a longitudinal direction along the intermediate section to vary in size the respective stabilizing section and the sealing section depending on positioning in the longitudinal direction of the intermediate marker band.

10. The system in accordance with claim 1, wherein the braided implantable device is independently freely rotatably relative to a delivery wire.

11. The system in accordance with claim 1, wherein the intermediate section allows for angular offset positioning between the stabilizing section and the sealing section.

12. A method for disrupting blood flow at a target site using an endovascular embolization system comprising a braided implantable device including: a folded over tubular braid forming a folded over atraumatic distal surface and an inner channel; wherein the folded over tubular braid is preformed into multiple distinct sections including: a stabilizing section including the folded over atraumatic distal surface; a sealing section disposed proximally of the stabilizing section; an intermediate section interposed directly between the stabilizing section and the sealing section; the intermediate section having a stiffness greater than that of the stabilizing section; and a proximal section disposed proximally of the sealing section; wherein each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the intermediate section and the proximal section; the stabilizing section comprises only a single braided layer while the sealing section comprises a plurality of nested layers providing enhanced stiffness greater than that of the stabilizing section while disrupting blood flow passable through open space regions defined by the plurality of nested layers; and wherein the folded over tubular braid forms the single braided layer of the stabilizing section and at least one of the plurality of nested layers of the sealing section; the method comprising the steps of:
navigating a microcatheter through a vasculature to the target site;
while in the radially constricted state, pushing in a distal direction the braided implantable device through the microcatheter using a delivery wire; and
upon exiting from a distal end of the microcatheter, deploying the braided implantable device at the target site; wherein when deployed the stabilizing section anchoring in position by radially expanding in direct physical contact with a vessel wall at the target site and together with the intermediate section pushing against and stabilizing in place at the target site the sealing section thereby minimizing risk of migration in a distal direction over time; the plurality of nested layers maximizing disruption of without preventing blood flow passable through the open space regions defined by the plurality of nested layers of the sealing section when deployed at the target site, while the stabilizing section having only the single braided layer maintains maximum compressibility and positioning when deployed at the target site.

13. The method in accordance with claim 12, wherein the deploying step further comprises the step of each of the stabilizing section and the sealing section independently self-adjusting in size and shape dependent on anatomy at the target site.

14. The method in accordance with claim 12, wherein the deploying step further comprises the step of repositioning the sealing section to be properly positioned at the target site with the folded over atraumatic distal surface of the stabilizing section acting as a bumper preventing damage to the vessel wall at the target site.

15. The method in accordance with claim 12, wherein the pushing step further comprises the step of minimizing twisting in a longitudinal direction of the braided implantable device by being independently freely rotatable relative to the delivery wire.

16. The method in accordance with claim 12, wherein the deploying step further comprises the step of permitting, via the intermediate section acting as a hinge, angular offset positioning of the sealing section while the stabilizing section is maintained anchored in place at the target site.

17. The method in accordance with claim 12, the deploying step further comprises the step of simultaneously self-adjusting in size the stabilizing section and the sealing section, respectively, based on positioning in a longitudinal direction of an intermediate marker band disposed about and freely slidable along the intermediate section.

18. The method in accordance with claim 12, wherein along one of the intermediate section, the proximal section, or the sealing section is secured each free edge of the single braided layer of the stabilizing section and each free edge of the plurality of nested layers of the sealing section.

19. The method in accordance with claim 12, wherein the plurality of nested layers of the sealing section include at least one supplemental structural obstruction layer separate from the folded over tubular braid; wherein the at least one supplemental structural obstruction layer is a supplemental braid layer, at least one supplemental coil, or at least one supplemental suture.

20. A method for manufacture of a braided implantable endovascular embolization device including: a folded over tubular braid forming a folded over atraumatic distal surface and an inner channel; wherein the folded over tubular braid is preformed into multiple distinct sections including: a stabilizing section including the folded over atraumatic distal surface; a sealing section disposed proximally of the stabilizing section; an intermediate section interposed directly between the stabilizing section and the sealing section; the intermediate section having a stiffness greater than that of the stabilizing section; and a proximal section disposed proximally of the sealing section; wherein each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the intermediate section and the proximal section; the stabilizing section comprises only a single braided layer while the sealing section comprises a plurality of nested layers providing enhanced stiffness greater than that of the stabilizing section while disrupting blood flow passable through open space regions defined by the plurality of nested layers; and wherein the folded over tubular braid forms the single braided layer of the stabilizing section and at least one of the plurality of nested layers of the sealing section; the method comprising the steps of:
providing a cylindrical braided tubular structure defining a passageway in a longitudinal direction between two free edges at opposite ends;
folding over on itself the cylindrical tubular braided structure with the two free edges extending in a same direction to form the folded over tubular braid having the folded over atraumatic distal surface and the inner channel;
arranging at least one supplemental structural obstructing layer and/or the detachment mechanism longitudinally in the inner channel of the folded over tubular braid;
preforming together the folded over tubular braid and/or at least one supplemental structural obstructing layer about the detachment mechanism into respective multiple distinct sections including: the stabilizing section; the intermediate section; the sealing section; and the proximal section; wherein the detachment mechanism is secured within the inner channel of the proximal section; and
securing in place only along one of the proximal section, the intermediate section, or the sealing section each free edge of the single braided layer of the stabilizing section and each free edge of the plurality of nested layers of the sealing section.
